(19) United States
(12) Patent Application Publication
(10) Pub. No.: US 2021/0068751 A1
(43) Pub. Date: Mar. 11, 2021

(54) AUTISM SUPPORT SYSTEM, WEARABLE DEVICE, AND METHODS OF USE

(71) Applicant: Kelsy HOLLE, San Diego, CA (US)

(72) Inventor: Kelsy HOLLE, San Diego, CA (US)

(21) Appl. No.: 17/013,428

(22) Filed: Sep. 4, 2020

Related U.S. Application Data
(60) Provisional application No. 62/896,691, filed on Sep. 6, 2019.

(51) Int. Cl. A61B 5/00; G16H 50/30

(52) U.S. Cl. CPC A61B 5/681; G16H 15/00; G16H 50/30; A61B 5/0022

(57) ABSTRACT

Systems and methods for an autism management and/or diagnosis support system are described. The autism management and/or diagnosis support system can include a computing device, including a wearable computing device, configured to receive an input from a user to select a client profile and display on a user interface the client profile. The computing device can be further configured to receive a second input from the user to record behavioral event data and begin recording the behavioral event data. The computing device can also retrieve historical behavioral data associated with the client profile and compile a summary of behavior associated with the client profile to be displayed for the user.

### CLAIM OF PRIORITY

This application claims priority and benefit under 35 U.S.C. § 119(e) of U.S. Provisional Patent Application 62/896,691, filed 9 Sep. 2019 which is fully incorporated herein by reference.

### FIELD

Examples of the present disclosure relate to a behavioral management and/or diagnosis support system, and, more particularly, to an autism management and/or diagnosis support system.

### BACKGROUND

Autism is a developmental disorder that can be observed in children as early as three years of age or younger. Autism is typically characterized by difficulties with social interaction and communication as well as restrictive or repetitive behavior. Symptoms of autism can develop gradually and sometimes cause worsening behavior or communication problems as a child grows. Behavioral and communication problems, however, can be improved with effective diagnosis and treatment.

One method of diagnosing and treating an individual with autism involves observing, collecting, and analyzing the individual's behaviors over time to develop an effective treatment plan. This method, commonly referred to as Applied Behavior Analysis (ABA), incorporates behavioral adaptation based on the principles of cause and effect to influence whether negative behaviors are repeated. For example, by observing, collecting, and analyzing an individual's behaviors over time, medical professionals and caregivers (e.g., pediatricians, board certified behavior analysts, therapists, psychologists, psychiatrists, registered behavior technicians, caregivers, etc.) are able to determine common antecedents, behaviors, and consequences (ABCs) of the individual's actions and determine appropriate ways to positively reinforce the individual's good behavior and discourage inappropriate behavior. By understanding the triggering function of an individual's behavior, medical professionals and caregivers using ABA can apply positive consequences to reinforce appropriate replacement behaviors. ABA identifies these triggering functions through behavior-focused data collection and pattern analysis.

Collecting and analyzing the individual's behavioral data, however, typically involves a medical professional, parent, teacher, or caregiver manually recording the child's behaviors on a paper and trying to trend the data over time, which can often be an arduous task. Because the task of collecting and analyzing the individual's behavioral data can be arduous, inaccurate data is often collected which can lead to misdiagnosis and ineffective treatment of those struggling with autism.

What is needed, therefore, is a system and method of effectively collecting and analyzing behavioral data to achieve improved diagnosis and treatment of those struggling with autism. These and other problems are addressed by the technology disclosed herein.

### SUMMARY

Accordingly, the inventors of this disclosure have recognized that there is a need for the following solution.

The disclosed technology can include a method of tracking and analyzing behavioral data using a computing device. The method can include receiving, at a processor associated with the computing device, a first input from a user to select a client profile and displaying, on a user interface, the client profile. The method can also include receiving, at the processor associated with the computing device, a second input from the user to record event data associated with a behavioral event of a client and recording, by the processor and to a memory associated with the computing device, the event data. The method can further include retrieving, by the processor and from the memory, historical behavioral data associated with the client profile, compiling, by the processor based on the event data and the historical behavioral data, a summary of behavior associated with the client profile; and displaying, on the user interface, the summary of behavior associated with the client profile.

Receiving the first input and receiving the second input can be performed by a processor associated with a wearable device. The computing device can include a touch screen configured to receive a touch input from the user and at least one of the first input and the second input can include the touch input from the user. The computing device can also include a sound recording device configured to receive a voice command from the user and at least one of the first input and the second input can be the voice command from the user.

Receiving the first input from the user can include selecting the client profile from a user-configurable client list that can include a plurality of client profiles. The event data can be time data indicative of a duration of the behavioral event or the event data can be a description of the client's behavior during the behavioral event.

The method can include outputting, by the processor and to a connected computing device, the summary of behavior associated with the client profile. The summary of behavior associated with the client profile can be a chart summarizing the client's behavior over time. The method can also include retrieving, by the processor and from the memory, behavioral plan data associated with one or more predetermined behavioral plans and compiling, by the processor and based on the event data, the historical behavioral data, and the behavioral plan data, one or more client-specific data-based positive behavioral intervention plans.

The method can also include receiving, by the processor and from a positioning system associated with the computing device, location data associated with the behavioral event, compiling, by the processor based on the event data and the location data, a summary of the behavioral event, and displaying, at the user interface, the summary of the behavioral event. In other examples, the method can include receiving, at the processor, a third input from the user to record additional event data, the additional event data can also be associated with the behavioral event of the client. The method can further include recording, in the memory, the additional event data, and compiling, by the processor and based on the event data, the historical behavioral data, and the additional event data, the summary of behavior associated with the client profile.

The third input from the user can include selecting a severity level from a user-configurable list of severity levels descriptive of a severity of the behavioral event. The third input from the user can also include selecting an antecedent event from a user-configurable list of antecedent events descriptive of a cause of the behavioral event. In other examples, the third input from the user can include an event-specific note of the user or the third input from the user can include selecting a consequence from a user-configurable list of consequences descriptive of a consequence of the behavioral event.

The disclosed technology can include an apparatus having a graphical user interface on a display. The graphical user interface can be configured to present to a user, information of a summary of behavior associated with a client profile. The apparatus can further include a processor in communication with the graphical user interface and configured to receive a first input from a user to select a client profile and receive a second input from the user to record a behavioral event comprising event data associated with the behavioral event. The processor can also be configured to retrieve, from a memory in communication with the processor, historical behavioral data associated with the client profile. The processor can compile a summary of behavior associated with the client profile based on the event data and the historical behavioral data and display, on the graphical user interface, the summary of behavior associated with the client profile.

The apparatus can be a wearable device. The apparatus can further include a touch screen configured to receive a touch input from the user and at least one of the first input and the second input can be the touch input from the user. The apparatus can also include a sound recording device that can receive a voice command from the user and at least one of the first input and the second input can be the voice command from the user.

Receiving the first input from the user to select a client profile can include selecting the client profile from a user-configurable client list. The user-configurable client list can be a plurality of client profiles.

The event data can include time data indicative of a duration of the behavioral event and/or a behavioral description. The behavioral description can be a description of the client's behavior during the behavioral event.

The processor can also output the summary of behavior associated with the client profile to a connected computing device. The summary of behavior associated with the client profile can be a chart summarizing the client's behavior over time. The processor can also retrieve, from the memory, behavioral plan data associated with one or more predetermined behavioral plan and compile, by the processor and based on the event data, the historical behavioral data, and the behavioral plan data, one or more client-specific data-based positive behavioral intervention plans. The processor can also be configured to display, on the graphical user interface, the one or more client-specific data-based positive behavioral intervention plans.

In other examples, the processor can be configured to receive, from a positioning system associated with the computing device, location data associated with the behavioral event and compile, based on the event data and the location data, a summary of the behavioral event. The processor can also be configured to display, at the user interface, the summary of the behavioral event. The processor can be further configured to receive a third input from the user to record additional event data, the additional event data can be associated with the behavioral event of the client. The processor can record, in the memory, the additional event data and analyze, by the processor and based on the event data, the historical behavioral data, and the additional event data, to identify one or more behavioral patterns related to a medical condition by a client associated with the client profile.

The third input from the user can include selecting a severity level from a user-configurable list of severity levels descriptive of a severity of the behavioral event. The third input from the user can also include selecting an antecedent event from a user-configurable list of antecedent events descriptive of a cause of the behavioral event. The third input from the user can also include an event-specific note of the user or selecting a consequence from a user-configurable list of consequences descriptive of a consequence of the behavioral event.

The disclosed technology can also include a system for tracking and analyzing behavioral data comprising a graphical user interface configured to receive an input from a user and display data associated with a client profile, a memory configured to store the data associated with a client profile, and a processor in communication with the graphical user interface and the memory. The processor can be configured to receive a first input from a user to select a client profile and a second input from the user to record event data associated with a behavioral event of a client. The processor can retrieve, from the memory, historical behavioral data associated with the client profile, and compile a summary of behavior associated with the client profile based on the event data and the historical behavioral data. The processor can also be configured to display, on the graphical user interface, the summary of behavior associated with the client profile.

The graphical user interface can be a wearable device. The graphical user interface can further include a touch screen configured to receive a touch input from the user and at least one of the first input and the second input can be the touch input from the user. The system further comprises a sound recording device in communication with the processor and configured to receive a voice command from the user, wherein at least one of the first input and the second input comprises the voice command from the user. Receiving the first input from the user to select a client profile can include selecting the client profile from a user-configurable client list and the user-configurable client list can include a plurality of client profiles. The event data can be time data indicative of a duration of the behavioral event or the event data can be a behavioral description including a description of the client's behavior during the behavioral event.

The system can further include a connected computing device, wherein the processor is further configured to output the summary of behavior associated with the client profile to the connected computing device. The connected computing device can be a remote server. Furthermore, the summary of behavior associated with the client profile can include a chart summarizing the client's behavior over time.

The processor can be further configured to retrieve, from the memory, behavioral plan data associated with one or more predetermined behavioral plans. The processor can also compile, based on the event data, the historical behavioral data, and the behavioral plan data, one or more client-specific data-based positive behavioral intervention plans and display, on the graphical user interface, the one or more client-specific data-based positive behavioral intervention plans.

The system can also include a positioning system in communication with the processor and the processor can be configured to receive, from the positioning system, location data associated with the behavioral event. The processor can also compile, based on the event data and the location data, a summary of the behavioral event and display, at the user interface, the summary of the behavioral event.

The processor can also be further configured to receive a third input from the user to record additional event data. The additional event data can be associated with the behavioral event of the client. The processor can record, in the memory, the additional event data and analyze, based on the event data, the historical behavioral data, and the additional event data, to identify one or more behavioral patterns related to a medical condition of a client associated with the client profile.

The third input from the user can include a selection of a severity level from a user-configurable list of severity levels descriptive of a severity of the behavioral event. The third input from the user can also include a selection from an antecedent event from a user-configurable list of antecedent events descriptive of a cause of the behavioral event or the third input can be an event-specific note of the user. The third input from the user can also be a selection of a consequence from a user-configurable list of consequences descriptive of a consequence of the behavioral event.

The present disclosure will be more fully understood from the following detailed description of embodiments thereof, taken together with the drawings.

### BRIEF DESCRIPTION OF THE DRAWINGS

While the specification concludes with claims, which particularly point out and distinctly claim the subject matter described herein, it is believed the subject matter will be better understood from the following description in conjunction with the accompanying drawings, in which like numerals indicate like structural elements and features in various figures. The drawings are not necessarily to scale, emphasis instead being placed upon illustrating principles of the disclosure. The figures depict one or more implementations of the inventive devices, by way of example only, not by way of limitation.

FIG. 1 is an illustration of a computing device for recording and analyzing behavioral data, according to an example of the present disclosure.

FIGS. 2A-2M illustrate example views of a graphical user interface of a computing device for recording and analyzing behavioral data, according to the present disclosure.

FIG. 3 illustrates a table of data collected by the computing device for recording and analyzing behavioral data, according to the present disclosure.

FIGS. 4A-4C illustrate a table of data collected by the computing device for recording and analyzing behavioral data, according to the present disclosure.

FIGS. 5A-5D illustrate charts of data collected by the computing device for recording and analyzing behavioral data, according to the present disclosure.

FIGS. 6A-6B illustrate a graph of data collected by the computing device for recording and analyzing behavioral data, according to the present disclosure.

FIG. 7 is a schematic diagram illustrating a system for recording and analyzing behavioral data, according to the present disclosure.

FIG. 8 is a schematic diagram illustrating a method for recording and analyzing behavioral data, according to the present disclosure.

FIG. 9 is a schematic diagram illustrating a method for recording and analyzing behavioral data, according to the present disclosure.

FIG. 10 is a schematic diagram illustrating a method for recording and analyzing behavioral data, according to the present disclosure.

FIG. 11 is a schematic diagram illustrating a method for recording and analyzing behavioral data, according to the present disclosure.

FIG. 12 is a flowchart of a method for recording and analyzing behavioral data, according to the present disclosure.

FIG. 13 is a flowchart of a method for recording and analyzing behavioral data, according to the present disclosure.

### DETAILED DESCRIPTION

The features of the presently disclosed solution may be economically manufactured or assembled by using one or more distinct parts and associated components which, may be assembled together for removable or integral application. Unless defined otherwise, all terms of art, notations and other scientific terms or terminology used herein have the same meaning as is commonly understood by one of ordinary skill in the art to which this disclosure belongs.

In some cases, terms with commonly understood meanings are defined herein for clarity and/or for ready reference, and the inclusion of such definitions herein should not necessarily be construed to represent a substantial difference over what is generally understood in the art.

As used herein, “a” or “an” means “at least one” or “one or more.” As used herein, the term “user”, “subject”, “end-user” or the like is not limited to a specific entity or person.

In this description, references to “one embodiment”, “an embodiment”, or “embodiments” mean that the feature or features being referred to are included in at least one embodiment of the technology. Separate references to “one embodiment”, “an embodiment”, or “embodiments” in this description do not necessarily refer to the same embodiment and are also not mutually exclusive unless so stated and/or except as will be readily apparent to those skilled in the art from the description. For example, a feature, structure, act, etc. described in one embodiment may also be included in other embodiments but is not necessarily included. Thus, the current technology can include a variety of combinations and/or integrations of the embodiments described herein.

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±20% of the recited value, e.g. “about 90%” may refer to the range of values from 71% to 99%. Furthermore, as used herein, the term “client” can refer to a patient, a student, a child, an adult, or any individual that a user of the disclosed technology wishes to record behavioral data about.

As will become apparent throughout this disclosure, the present invention is directed to a device and method for recording and analyzing behavioral data. The behavioral data can be associated with a client such as a child, a student, a patient, or any other individual of which a user would like to record behavioral data. The disclosed technology can make it possible for a medical professional or caregiver to view and analyze the behavioral data to identify one or more behavioral patterns related to the client's medical condition. Furthermore, by identifying one or more behavioral patterns related to the client medical condition, the disclosed technology can facilitate creating a data-based positive behavioral intervention plan for the client that can help the client learn appropriate replacement behavior.

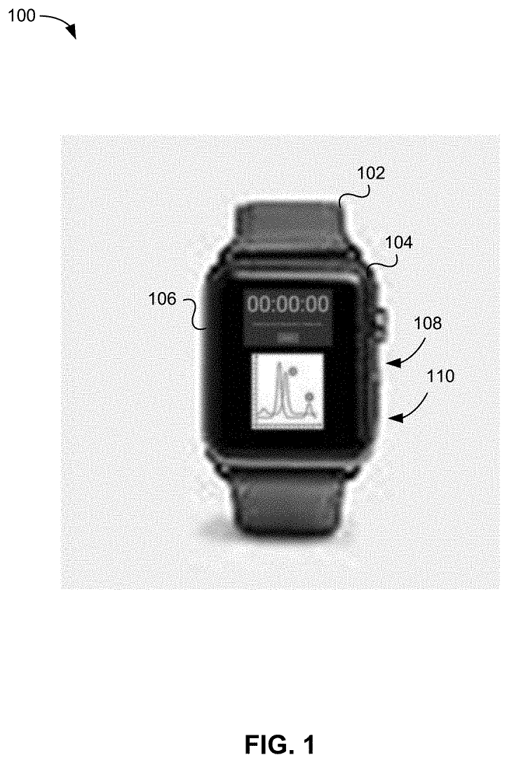

Referring now to the drawings, in which like numerals represent like elements, examples of the present disclosure are herein described. FIG. 1A illustrates a computing device 100 for recording and analyzing behavioral data, according to an example of the present disclosure. The computing device 100 can be, for example, a wearable device that is configured to be worn by a user, receive inputs from a user, determine actions based on received inputs, and display data to the user. Alternatively, the computing device 100 can be a handheld computing device, a laptop, a desktop, or any other computing device capable of receiving inputs, determining actions based on the inputs, and displaying data to the user. If the computing device 100 is a wearable device, the computing device 100 can include a wearable strap 102, such as a wrist band or other similar device configured to attach the computing device 100 to the user's wrist. As an example, the computing device 100 can be a smart watch configured to be worn by a user such that the user can easily access and interact with the computing device 100. As will be appreciated, by being wearable, the computing device 100 can be easily transported or carried by a user. This can be particularly helpful in cases where the user is, for example, a medical professional or caregiver attending to the needs of a client, such as a child, experiencing a behavioral event.

As will become apparent throughout this disclosure, the computing device 100 is configured to facilitate accurate and convenient recording of data associated with a client's behavior such that a user can contemporaneously record data associated with a behavioral event of a client as the behavioral event occurs. As used here, the behavioral event can include any behavior of an individual about which a user would be interested in recording data. For example and not limitation, a behavioral event can be or include a temper tantrum, aggression, injury to self, injury to others, eloping, non-compliance, social withdrawal, insistence on sameness, sensitivity to noises, sleep problems, mood changes, sudden change in activity, restrictive and/or repetitive behavior, or any other behavior exhibited by an individual about which a user would like to record data. By contemporaneously recording data during a behavioral event, the user can record the most accurate and up-to-date data that can be used to diagnose and treat an individual struggling with behavioral issues.

The computing device 100 can include a display 104 configured to display information and/or data associated with the behavioral event as well as other data related to a client's profile and data comprising various behavioral plans. The display 104 can include a graphical user interface 106 configured to receive an input from a user. The graphical user interface 106, for example, can be a touch screen configured to receive an input from a user. The input can be indicative of a user selecting a displayed option. (e.g., a client's name from a user-configurable client list). Thus, the display 104 can display an option or a list of options and the graphical user interface 106 can receive an input when the user has selected an option. As will be appreciated, the graphical user interface 106 can receive any input from a user as would be suitable for the particular application.

The computing device 100 can include a sound recording device 108 configured to detect sounds, such as a user's voice, and convert the sound to digital audio data. The sound recording device 108, for example, can be a microphone. The computing device 100 can be configured to receive voice commands from a user via the sound recording device 108 and perform certain actions based on the voice commands. As will be appreciated, by receiving voice commands and performing certain actions based on the voice command, the computing device 100 can advantageously be a hands-free device making it easier for the user to record behavioral data while remaining available to assist a client during a behavioral event. For example, the user can speak voice commands to the computing device 100 to begin recording behavioral data while simultaneously providing assistance to a client who exhibits inappropriate behavior during a behavioral event.

The computing device 100 can include a positioning system 110 configured to determine a location of the computing device 100. The positioning system 110 can be a local positioning system or a global positioning system (GPS) depending on the application. The positioning system 110 can be used, for example, to determine a location where a behavioral event of a client was observed. The computing device 100 can then record the location of the behavioral event. As will be appreciated, those struggling with behavioral issues can experience behavioral events more frequently based on their surroundings. The location data provided by the positioning system 110 and recorded by the computing device 100 can be particularly helpful for a user to determine locations where a client is more likely to experience behavioral events.

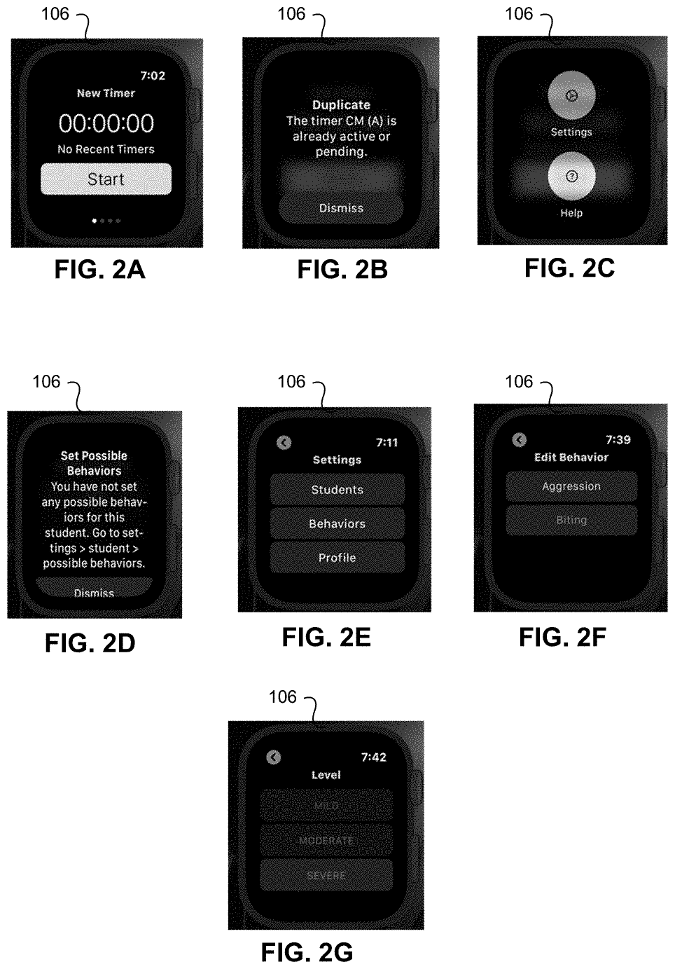

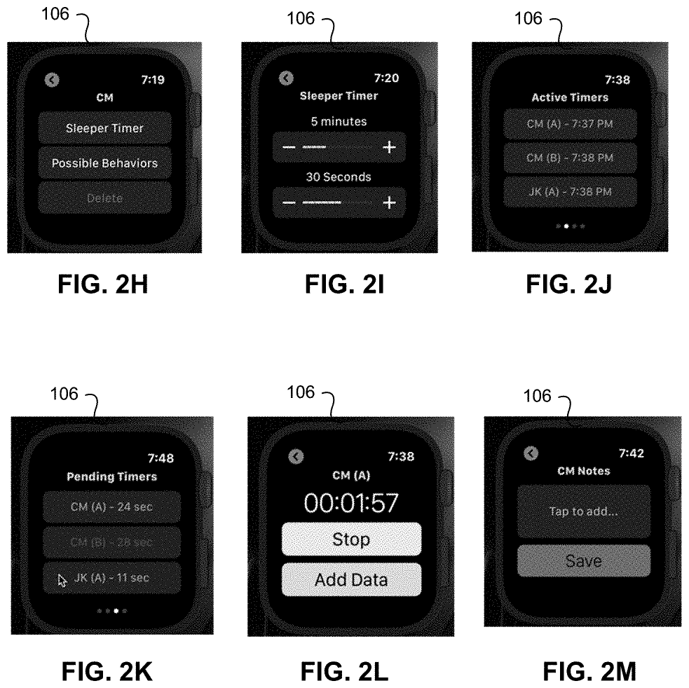

FIGS. 2A-2M illustrate example views of a graphical user interface 106 of the computing device 100 for recording and analyzing behavioral data, according to the present disclosure. As will be appreciated, the various example views described herein are offered for illustrative purposes and the present disclosure is not limited to the views described herein. FIG. 2A depicts an example view of a timer function of the computing device 100. For example, the computing device 100 can include a timer function that can, upon receiving an input such as a touch input or a voice command from a user, record a start time, a stop time, and a duration of a behavioral event. As depicted in FIG. 2B, the computing device 100 can include a status message to remind the user that a timer has already been started for a particular client or a particular event.

As depicted in FIG. 2C, the computing device 100 can be configured to include a settings menu and a help menu. The settings menu can allow a user to configure various settings of the computing device 100 (e.g., a list of clients, a client's profile, a list of behaviors, a list of severity of behaviors, a user's profile, etc.). For example, and as depicted in FIG. 2D, the graphical user interface 106 can output a message instructing the user to go to the settings menu, select a particular student profile, and then select possible behaviors descriptive of a student's behavior.

FIG. 2E depicts an example view of a settings menu of the computing device 100. As shown in FIG. 2E, the computing device 100 can allow a user to configure a list of clients (shown as “students” in FIG. 2E). For example, if the user is a teacher or other professional in an education setting, the settings menu can include a “students” option for a user to configure a list of students that the user is currently working with. The settings menu can also include options for the user to configure a list of behaviors and for the user to configure his or her own profile as desired. As will be appreciated, the settings menu can include settings other than those described herein.

FIG. 2F depicts a view of the settings options in which the user is able to configure a list of behaviors that can be descriptive of a client's behavior. By allowing a user to configure a list of behaviors, a user can customize the computing device 100 to be most useful for the user and the clients he or she is working with. Thus, the user can configure a list of behaviors that best suits the clients the user is working with.

FIG. 2G depicts a view of the of the settings options where a user is able to select and/or configure a severity of the behavior observed. For example, the user can observe a client's behavior and select a severity level that best describes the behavioral event. In this way, the user is able to not only track a client' behavior but also the severity level of the client's behavior. The computing device 100 can also be configured by the user to include additional severity levels than those depicted in FIG. 2G.

FIG. 2H depicts a view of the graphical user interface 106 displaying a list of options that can be available once a given timer has been started by a user to record data about a behavioral event. The list of options can include, but is not limited to, “sleeper timer,” “possible behaviors,” and “delete.” If a user selects “sleeper timer,” the user can adjust the duration of time a sleeper timer is active via the user interface as depicted in FIG. 2I.

As depicted in FIG. 2J, the computing device 100 can be configured to record data of multiple clients at a single time by having multiple timers simultaneously recording data. Furthermore, as depicted in FIG. 2K, the graphical user interface 106 can be configured to change colors for a given timer to indicate to the user that certain timers are stopped while others are actively recording.

The graphical user interface 106 can include an option to add data (as depicted in FIG. 2L) in which the user can add data to a given timer that can be descriptive of the behavioral event. For example, and not limitation, the additional data can include a description of the behavior associated with the behavioral event, a severity level of the behavioral event, an antecedent associated with the behavioral event, a consequence associated with the behavioral event, or even additional custom notes entered by the user (as depicted in FIG. 2M). As will be appreciated, the computing device 100 can be configured to record data about a client's behavioral event that can then be used to determine appropriate data-based behavior intervention plans to help the client correct inappropriate behavior. The data can be stored in a data table and used by the computing device to output one or more graphs or charts summarizing the data such that a user can determine behaviors common to the client, antecedents that often lead to a client's given behavior, and consequences that often follow a client's given behavior. By quickly and accurately compiling and summarizing information related to a client's behavior, a user can determine, based on the behavioral data, triggering functions and patterns related to the client's behavior and appropriate actions that can help teach the client appropriate replacement behavior based on behavioral intervention plans.

Alternatively, or in addition, the computing device 100 can be configured to store data related to behavioral intervention plans such that the computing device 100 can develop behavioral intervention plans tailored to a specific client based on the client's behavioral data. For example, the computing device 100 can store data related to predetermined behavioral intervention plans and, based on a client's behavioral data, determine what behavioral intervention plan would best fit a particular client based on the behavioral data. The user can further configure the computing device 100 to customize behavioral intervention plans to better fit a particular client's behavior. For example, the computing device 100 can output a determined behavioral intervention plan and, upon reviewing the behavioral intervention plan, the user can customize the behavioral intervention plan to best fit the particular client's situation.

The computing device 100 can be further configured to store historical behavioral data associated with a client's profile. For example, a user can cause the computing device 100 to record multiple instances of a client's behavioral events and store the data associated with the multiple instances of a client's behavioral events. In this way, the computing device 100 can store historical behavioral data associated with a client's behavioral events. Furthermore, the computing device 100 can be configured to trend the historical behavioral data over time and display the historical behavioral data on the display 104 such that a user can see a client's behavior over time and determine the client's progress. The computing device 100 can be further configured to determine a behavioral intervention plan based on the client's historical behavioral data and, after trending the historical behavioral data over time, adjust the client's behavioral intervention plan to inform the user how to best teach the client appropriate replacement behavior.

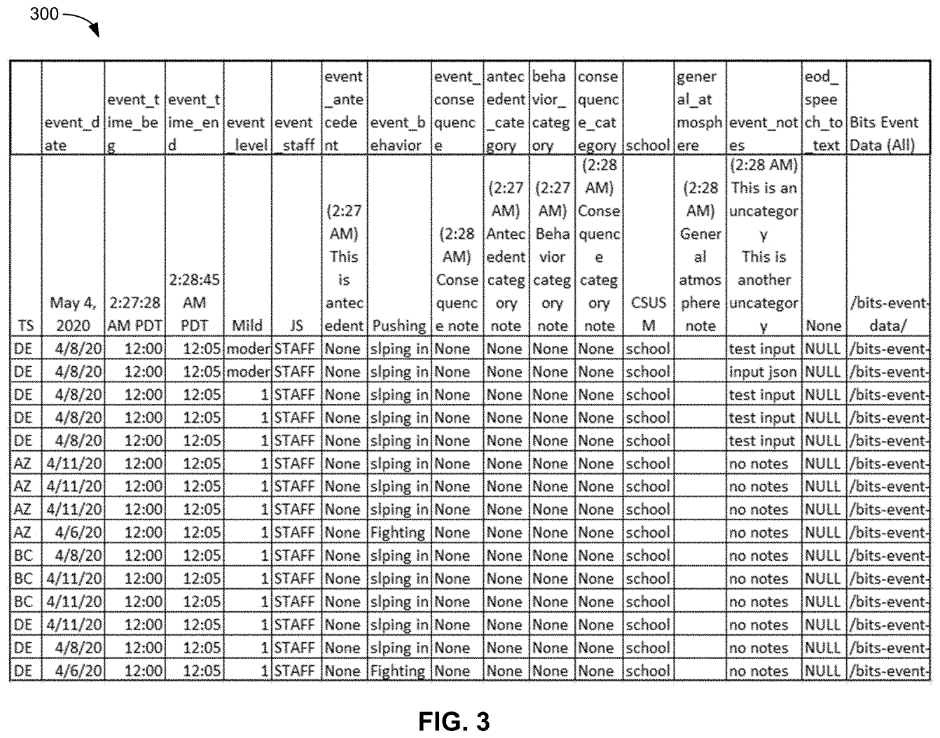

The historical behavioral data can be stored and displayed in a data table 300 as depicted in FIG. 3. The data table 300 can include all information relating to behavioral events that have been recorded. For example and not limitation, the data table 300 can include, starting from the left side of the data table, a title associated with the client, the client or student's identification (e.g., the client's name, the client's identification number, the client's nickname, etc.), the date of the behavioral event, a beginning time of the behavioral event, an ending time of the behavioral event, a severity level of the behavioral event, an indication of who recorded the data (event staff), an antecedent to the behavior, a description of the behavior, a consequence of the behavior, a category of the antecedent, a category of the behavior, a category of the consequence, a school or other institution name, a description of the general atmosphere, an indication of whether the event was recorded by receiving voice inputs from the user, and other event data as inputted by the user to the computing device 100. The data table 300 can be used by a user to review the historical data. Alternatively, or in addition, the data table 300 can be used by the computing device to compile the data into charts or graphs that can be more easily observed by the user.

The computing device 100 can be in communication with a connected computing device (e.g., a desktop, a laptop, a handheld computing device, a server, etc.) such that the computing device 100 can output the behavioral data and the historical behavioral data to the connected computing device.

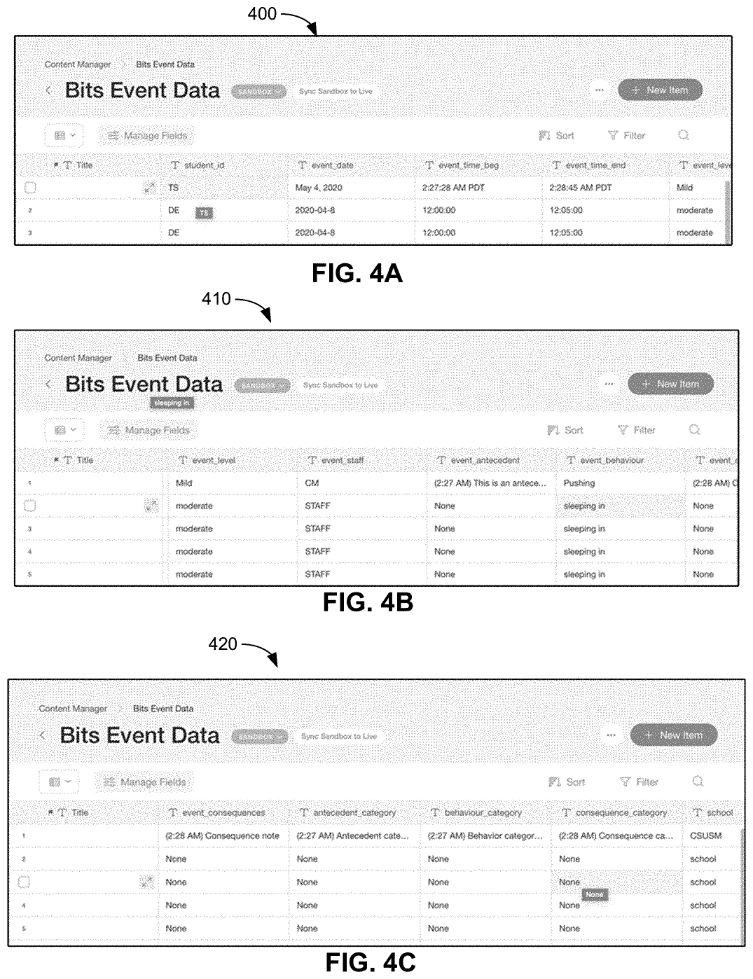

FIGS. 4A-4C illustrate an alternative example of the data table of FIG. 3. The data tables depicted in FIGS. 4A-4C can be, for example, a view of the data included in the data table as would be viewed and managed on a connected computing device. The data tables depicted in FIGS. 4A-4C can include the same data described in relation to FIG. 3 and be organized in the same order as the data described in FIG. 3. For example, the data depicted in data table 300 can correlate to the data shown in data table 400, data table 410, and data table 420. As will be appreciated, data tables 400, 410, and 420 can be, for example, data from the data table 300 compiled in the same data table but unable to be viewed on a single screen because the width of the data table is too large to view on a single screen. For example, FIG. 4A can depict a first portion of the data table as viewed on a connected computing device, FIG. 4B can depict a second portion of the data table as viewed on a connected computing device, and FIG. 4C can depict a third portion of the data table as viewed on a connected computing device. By outputting the data of the data table 300 to a connected computing device, the data contained in the data table can be more easily viewed and managed than would otherwise be, for example, on a wearable computing device. Furthermore, the data contained in the data table can be more easily managed and organized according to the user's preferences.

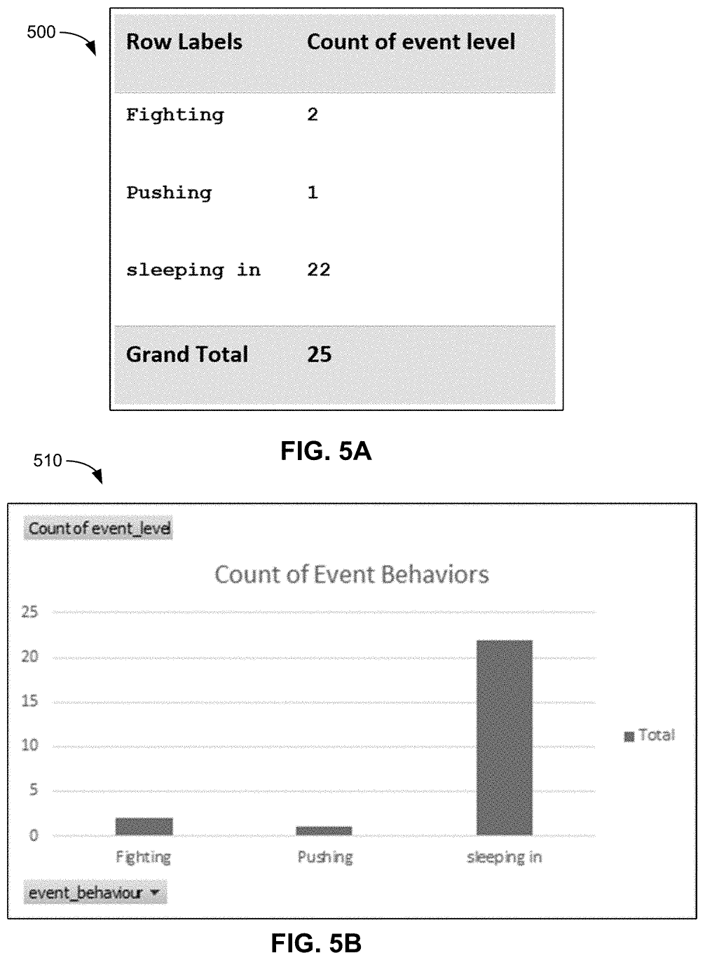

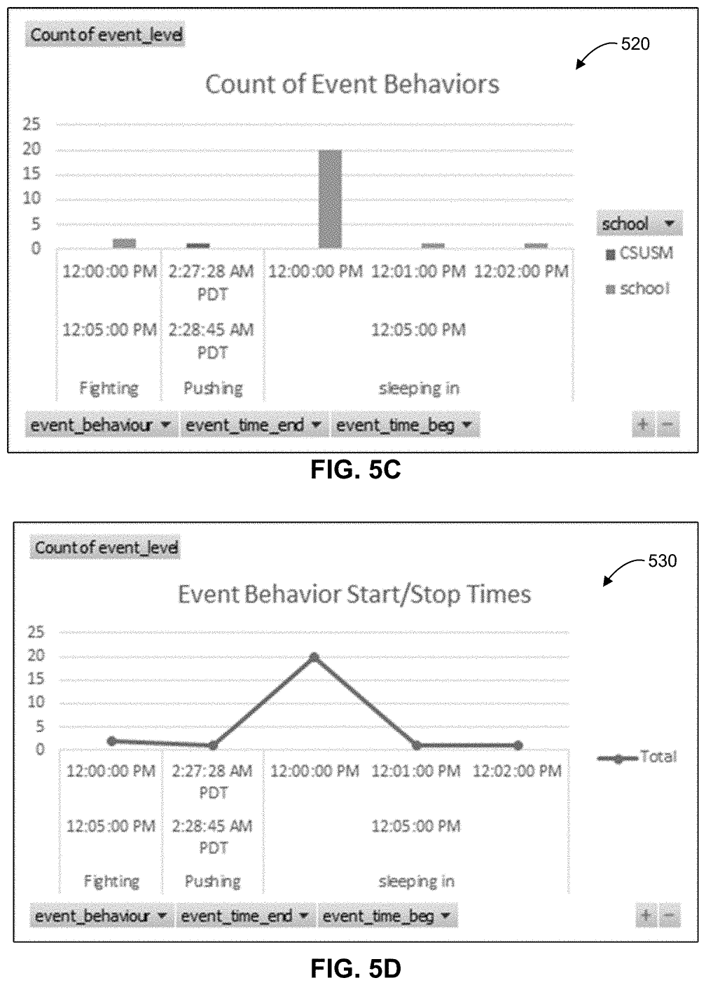

FIGS. 5A-5D illustrate charts of data collected by the computing device 100 for recording and analyzing behavioral data, according to the present disclosure. As will be appreciated, the behavioral data collected by the computing device 100 can be organized and summarized in various ways to display the behavioral data as would be preferred by the user. For example, the behavioral data can be summarized in a pivot table 500 as depicted in FIG. 5A. The pivot table 500 can include a behavior and a corresponding number of times that particular behavior has been recorded. Furthermore, the pivot table 500 can include a total number representing the total number of times the client has experienced a behavioral event. As another example, the behavioral data can be compiled into a bar graph 510 (as depicted in FIG. 5B) depicting a visual comparison of the total number of behavioral events. The behavioral data can also be compiled into a bar graph 520 (as depicted in FIG. 5C) depicting the behavior, the duration of the behavior, and the location (or “school”) where the behavior was observed. As will be appreciated, the location can be determined by data obtained by the positioning system 110 or, alternatively, by location data manually entered by the user. Alternatively, or in addition, the behavioral data can be compiled into a line graph 530 (as depicted in FIG. 5D) depicting a total number of times a behavior was observed during a particular time of day. This can be useful, for example, to help the user determine specific triggering events that lead to behavioral issues and to determine an appropriate behavioral intervention plan for the client that can include details of actions for the client depending on the time of day.

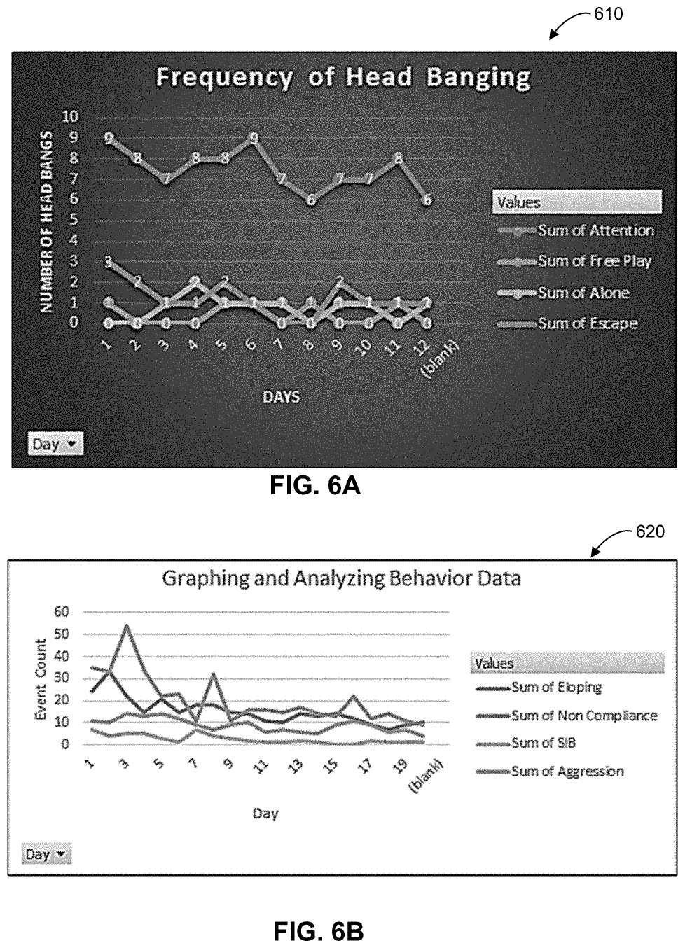

FIGS. 6A-6B illustrate graphs of behavioral data collected by the computing device 100, according to the present disclosure. As depicted in FIG. 6A-6B, the behavioral data collected by the computing device 100 can be compiled into a graph showing the tracked behavioral events over time. The behavioral data can be compiled into a line graph 610 or 620 that visually depicts a change in the client's behavior over time. As will be appreciated, by viewing the client's behavior over time, a user can more accurately determine a client behavior and the client's progress over time and adjust a behavioral intervention plan to effectively help a client change his or her behavior. The line graphs 610 and 620 can include individual lines corresponding to specific behaviors observed and recorded by the user via the computing device 100. Thus, a user can observe a change in a specific behavior over time to determine a client's progress with that specific behavior.

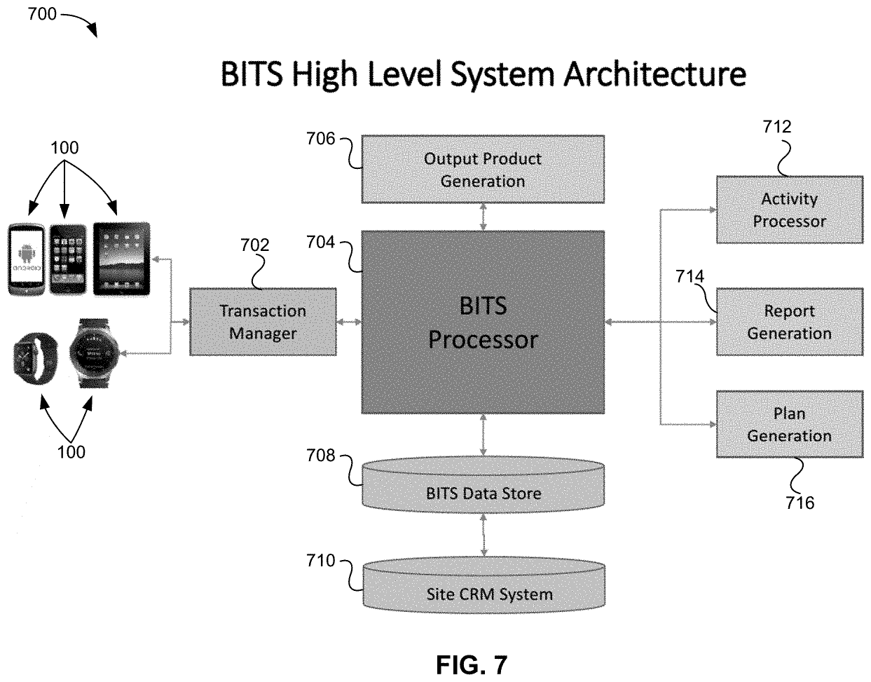

FIG. 7 is a schematic diagram illustrating a system 700 for recording and analyzing behavioral data, according to the present disclosure. The system 700 can include the computing device 100 which can be, for example, a handheld computing device or a wearable computing device. The system 700 can be completely contained in the computing device 100 or the system 700 can include additional components in communication with the computing device 100. For example, the computing device 100 can include an application or program configured to facilitate recording, trending, and displaying behavioral data directly on the computing device 100 and/or cause the computing device 100 to be in communication with a remote computing device or server to record, trend, and display the behavioral data.

The system 700 can include a transaction manager 702 configured to control coordination of transactions between the computing device 100 and the processor 704, or between the graphical user interface 106 and the processor 704. The system 700 can include a processor 704 configured to receive inputs, determine appropriate actions based on the inputs, and output data. The processor 704, for example, can be configured to record, trend, and compile behavioral data based on inputs received from the user. Furthermore, the processor 704 can be configured to output product generation 706 such as compiled behavioral data. The output product generation 706 can be, for example, the tables, charts, and/or graphs previously described in relation to FIG. 3-FIG. 6B.

The processor 704 can be in communication with a memory 708 such as the BITS Data Storage depicted in FIG. 7. The processor 704 can be configured to send data to the memory 708 or retrieve data from the memory 708 depending on the specific action. The memory 708 can be configured to receive and store data associated with a behavioral event. Furthermore, the memory 708 can be in communication with a site customer relationship management (CRM) system 710. The site CRM system 710 can receive data from, and send data to, the memory 708 as necessary to ensure the system 700 is capable of most effectively recording, storing, and analyzing behavioral data. For example, the site CRM system 710 can be configured to receive data relating to behavioral plans such that behavioral plans can be customized and revised as necessary for ensuring the behavioral plans remain up-to-date and are effective in helping to correct a client's behavior.

The processor 704 can further be configured to either be in communication with additional processors such as an activity processor 712, a report generation processor 714, and/or a plan generation processor 716 or operate independently. As an example, if the processor 704 is contained in the computing device 100, the processor 704 can be configured to output data and/or instructions to a remote processor (e.g., activity processor 712, a report generation processor 714, and/or a plan generation processor 716), to perform certain actions. Alternatively, the processor 704 can be configured to perform the functions of the activity processor 712, a report generation processor 714, and/or a plan generation processor 716. The activity processor 712 can be configured to receive behavioral data and perform analysis of the behavioral data to determine a client's behavior. The report generation processor 714 can be configured to generate, based on the behavioral data, one or more reports depicting a summary of the behavioral data collected. As will be appreciated, the report generation processor 714 can be configured to compile and output any of the tables, charts, and/or graphs previously described in relation to FIG. 3-FIG. 6B. Alternatively, or in addition, the processor 704 can be configured to perform the same report generation functions just described in relation to the report generation processor 714.

The plan generation processor 716 can be configured to compile one or more data-based positive behavioral intervention plans that can be implemented by the user, a parent, a medical professional, a caregiver, or any individual or organization working with a client to help the client learn appropriate replacement behavior. The plan generation processor 716 can be configured to receive the behavioral data and analyze the behavioral data in comparison to predetermined behavioral intervention plan data to determine a positive behavioral intervention plan that would best fit the particular client. As previously described, the user can further configure the behavioral intervention plan data such that a behavioral intervention plan can be customized for a particular client. Alternatively, or in addition, the processor 704 can be configured to perform the same behavioral intervention plan generation functions just described in relation to the plan generation processor 716.

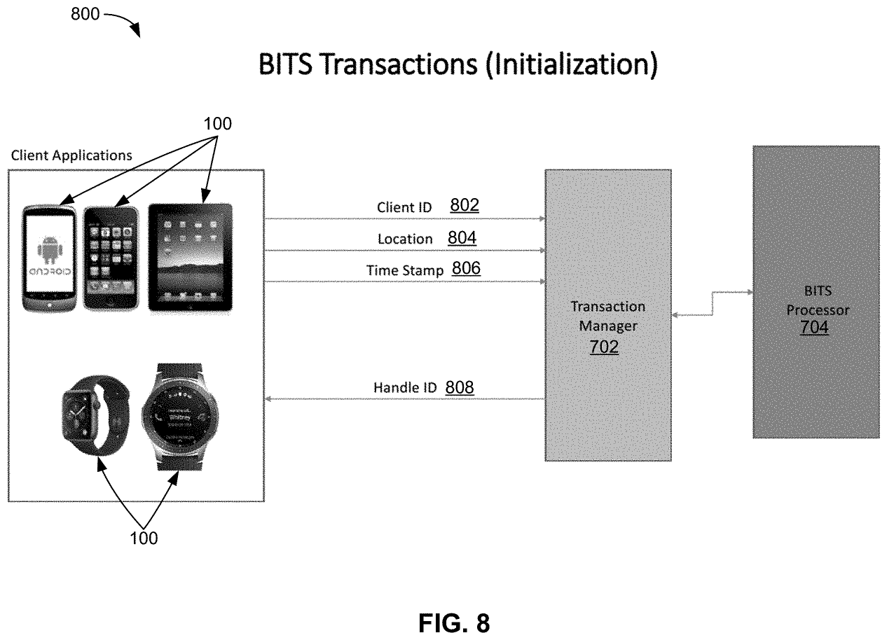

FIGS. 8-10 are schematic diagrams illustrating a method 800 for recording and analyzing behavioral data, according to the present disclosure. The method 800 described herein can correspond to a user interacting with the computing device 100 and the computing device performing certain actions based on the inputs received from the user. As depicted in FIG. 8, the method 800 can include initialization that can correspond to a user recording a behavioral event. For example, upon the user selecting to begin recording a behavioral event, the method 800 can include the computing device sending a client identification (ID) 802, a location 804, and a time stamp 806 data, to the transaction manager 702. The transaction manager can then send the client identification (ID) 802, the location 804, and the time stamp 806 data to the processor 704 to begin recording the behavioral event. The transaction manager 702 can also send a handle identification (ID) 808 to the computing device 100 to help manage recording the behavioral event.

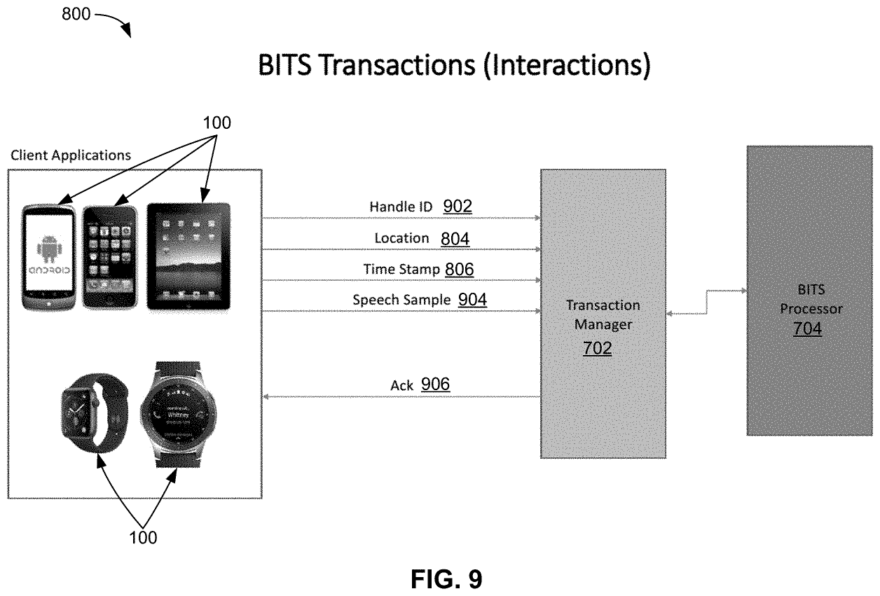

After initializing a session recording the behavioral data, the method 800 can further include, as depicted in FIG. 9, the user inputting additional data to record additional behavioral data. For example, the method 800 can include sending the handle identification 902 to the transaction manager along with the location 804, the time stamp 806 and a speech sample 904. The speech sample 904 can be a voice command received at the computing device 100 via the sound recording device 108. The transaction manager 702 can send the received data to the processor 704. Furthermore, the transaction manager 702 can send an acknowledgement 906 to the computing device 100 to inform the user that the data (e.g., the voice command) has been received.

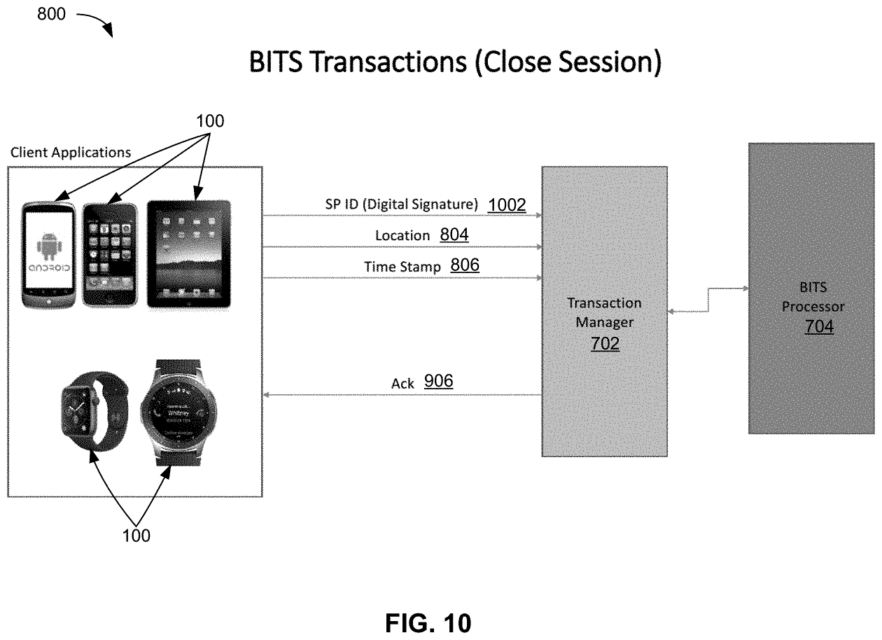

Once the user has recorded the behavioral data and is ready to cease recording behavioral data, the method 800 can include, as depicted in FIG. 10, ending the session by the computing device 100 sending a digital signature 1002 along with the location 804 and a time stamp 806 to the transaction manager 702 to end the session. The transaction manager can send the received data to the processor 704 and send an acknowledgement 906 to the computing device 100 so the user knows the command was received.

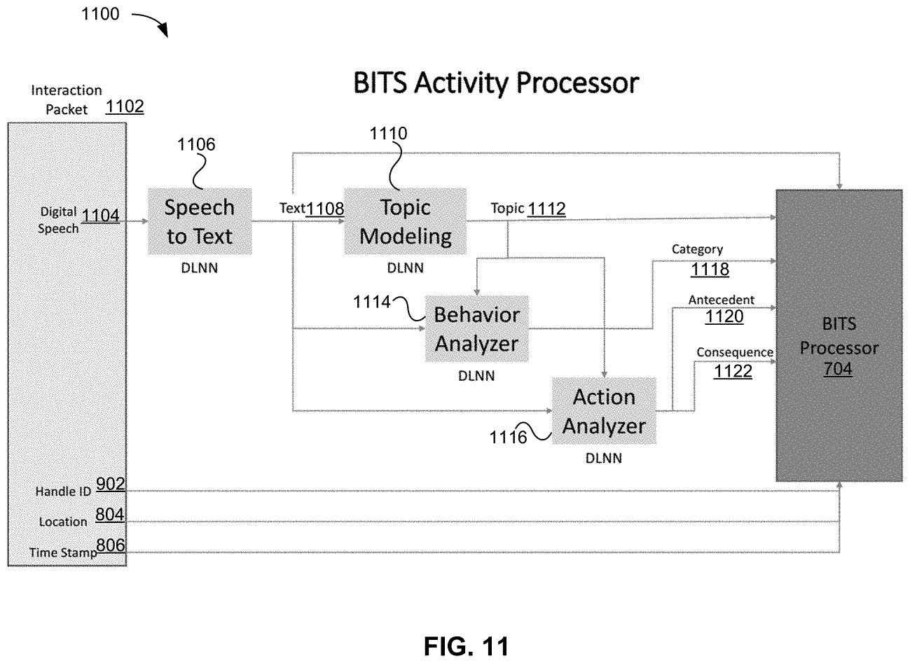

FIG. 11 is a schematic diagram illustrating a method 1100 for recording and analyzing behavioral data, according to the present disclosure. The method 1100 can include an interaction packet 1102 that can be a packet of data comprising digital speech 1104 (e.g., voice data captured by the sound recording device 108 and converted to digital speech data, or speech to text data 1106), a handle identification (ID) 902, location data 804, and a time stamp 806. The speech to text data 1106 can be converted using a dynamic learning neural network (DLNN). The speech to text data 1106 can then be directed to the processor 704. Alternatively, or in addition, the speech to text data 1106 can be directed as text 1108 to a topic modeling DLNN 1110, a behavior analyzer DLNN 1114, and/or an action analyzer DLNN 1116. The topic modeling DLNN 1110 can be configured to analyze the speech to text data 1106 and learn specific topics related to behaviors of a client as described by the user. The data from the topic modeling DLNN can output topic data to the processor 704, the behavior analyzer DLNN 1114, and/or the action analyzer DLNN 1116.

As mentioned previously, the speech to text data 1106 can be sent to the behavior analyzer DLNN 1114. The behavior analyzer DLNN 1114 can be configured to analyze the speech to text data 1106 and learn specific behaviors of a client as described by the user. The behavior analyzer DLNN 1114 data can then be assigned a category 1118 and outputted to the processor 704.

The speech to text data can also be sent to the action analyzer DLNN 1116. The action analyzer DLNN 1116 can analyze the speech to text data 1106 and determine a corresponding antecedent 1120 and a corresponding consequence 1122 as described by the user. The antecedent data 1120 and the consequence data 1122 can then be outputted to the processor 704.

Upon receiving the speech to text data 1106, the topic data 1112, the category data 1118, the antecedent data 1120, and the consequence data 1122, the processor 704 can record and analyze the data. The processor 704 can further compare the speech to text data 1106, the topic data 1112, the category data 1118, the antecedent data 1120, and the consequence data 1122, the processor 704 to historical behavioral data and positive intervention plan data to compile a report of the client's behavior. The report can include the same or similar tables, charts, and graphs previously described as well as a related behavioral intervention plan selected to help the client learn appropriate replacement behavior.

As will be appreciated, the topic data 1112, the category data 1118, the antecedent data 1120, and the consequence data 1122, can each relate to elements of information that is generally used for Applied Behavior Analysis (ABA), including the antecedent, behavior, and consequences (ABC). The data-based positive behavioral intervention plan can correlate to intervention plans commonly used for ABA and can be specifically tailored to the client based on the ABC data (e.g., the topic data 1112, the category data 1118, the antecedent data 1120, and the consequence data 1122) sent to, and analyzed by, the processor 704.

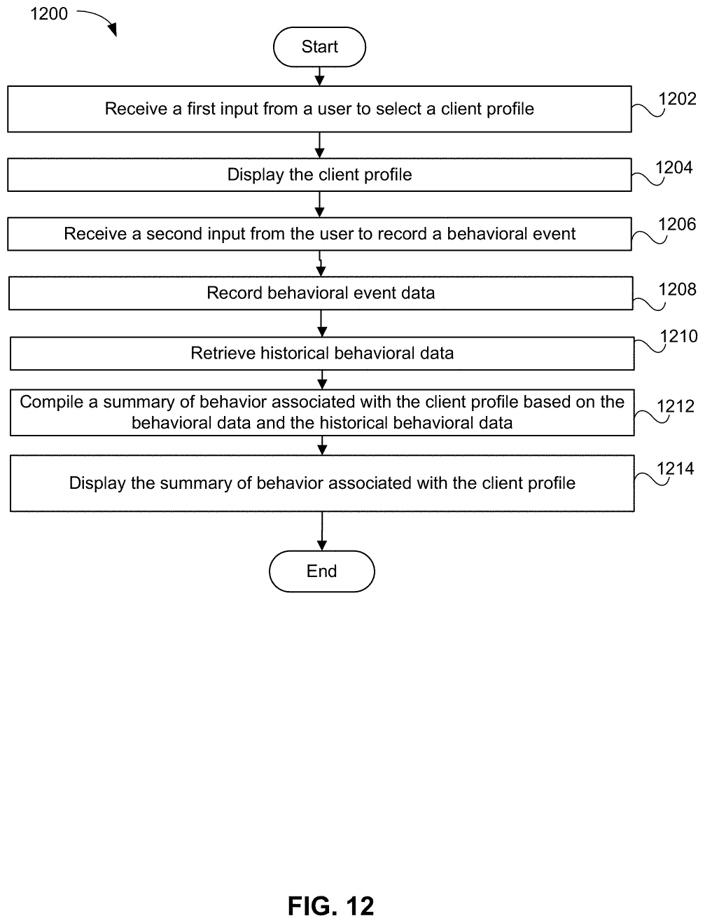

FIG. 12 is a flowchart of a method 1200 for recording and analyzing behavioral data, according to the present disclosure. The method 1200 can include receiving 1202 a first input from a user to select a client profile and displaying 1204 the client profile. The method 1200 can further include receiving 1206 a second input from the user to record a behavioral event and recording 1208 the event data. As will be appreciated, the event data can comprise any of the behavioral data described herein, including, but not limited to, a description of a behavioral event, a duration of a behavioral event, an antecedent of a behavioral event, a consequence of a behavioral event, a location of a behavioral event, etc. The method 1200 can further include retrieving 1210 historical behavioral data and compiling 1212 a summary of behavior associated with the client profile based on the behavioral data and the historical behavioral data. The method 1200 can then include displaying the summary of behavior associated with the client profile 1214.

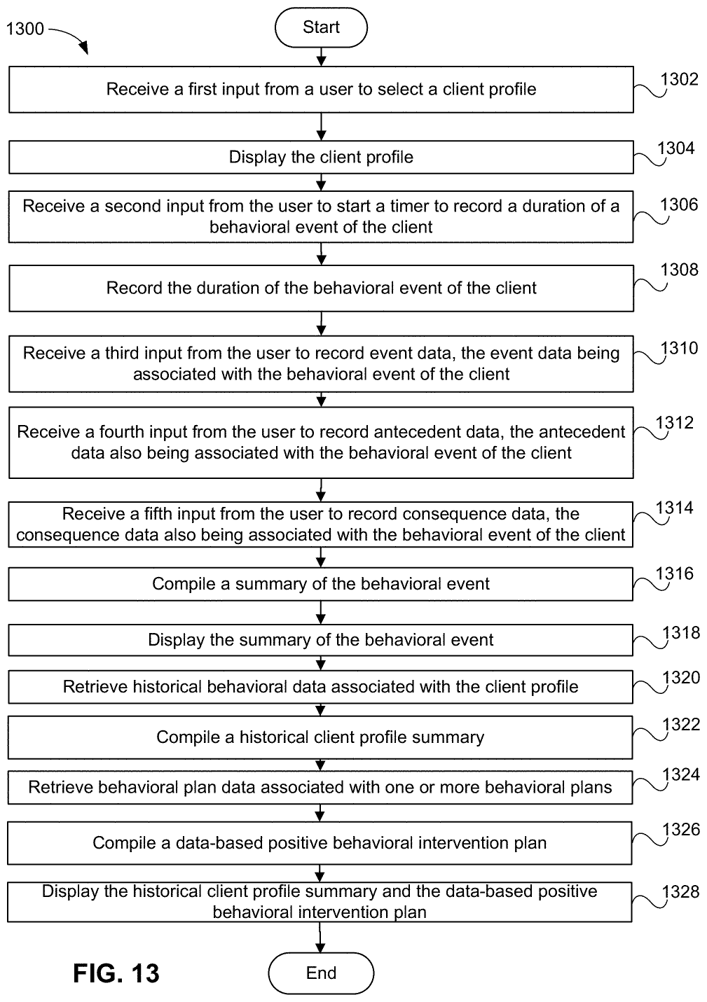

FIG. 13 is a flowchart of a method 1300 for recording and analyzing behavioral data, according to the present disclosure. The method 1300 can include receiving 1302 a first input from a user to select a client profile and displaying 1304 the client profile. The method 1300 can further include receiving 1306 a second input from the user to start a timer to record a duration of a behavioral event of the client and recording 1308 the duration of the behavioral event of the client. The method 1300 can include receiving 1310 a third input from the user to record event data associated with the behavioral event of the client. As will be appreciated, the event data can comprise any of the behavioral data described herein, including, but not limited to, a description of a behavioral event, an antecedent of a behavioral event, a location of a behavioral event, etc.

The method 1300 can include receiving 1312 a fourth input from the user to record antecedent data that can also be associated with the behavioral event of the client. The method 1300 can further include receiving a fifth input 1314 from the user to record consequence data associated with the behavioral event of the client. As will be appreciated, the antecedent data can correspond to a description of an antecedent that caused, or is related to the cause of, the behavioral event. Similarly, the consequence data can correspond to a description of a consequence of the behavioral event.

The method 1300 can include compiling a summary of the behavioral event 1316 and displaying the summary of the behavioral event 1318. The summary of the behavioral event can be based on the client profile, the duration of the behavioral event, the event data, the antecedent data, and the consequence data. The method can also include retrieving 1320 historical behavioral data associated with the client profile and compiling 1322 a historical client profile summary. The historical client profile can be based on the client profile, the duration of the behavioral event, the event data, the antecedent data, the consequence data, and the historical behavioral data.

The method 1300 can also include retrieving 1324 behavioral plan data associated with one or more behavioral plans and compiling 1326 a data-based positive behavioral intervention plan. The data-based positive behavioral intervention plan can be based on the client profile, the duration of the behavioral event, the event data, the antecedent data, the consequence data, the historical behavioral data, and the one or more behavioral plans. The method 1300 can further include displaying 1328 the client profile summary and the data-based positive behavioral intervention plan.

As will be appreciated, the methods 1200 and 1300 just described can be varied in accordance with the various elements and examples described herein. That is, methods in accordance with the disclosed technology can include all or some of the steps described above and/or can include additional steps not expressly disclosed above. Further, methods in accordance with the disclosed technology can include some, but not all, of a particular step described above. As non-limiting examples, although not described in methods 1200 and 1300, each of the methods can include additional elements described herein such as any of the components or method steps mentioned herein.

The definitions of the words or elements of the following claims are, therefore, defined in this specification to not only include the combination of elements which are literally set forth. It is also contemplated that an equivalent substitution of two or more elements can be made for any one of the elements in the claims below or that a single element can be substituted for two or more elements in a claim. Although elements can be described above as acting in certain combinations and even initially claimed as such, it is to be expressly understood that one or more elements from a claimed combination can in some cases be excised from the combination and that the claimed combination can be directed to a subcombination or variation of a subcombination(s).

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements. The claims are thus to be understood to include what is specifically illustrated and described above, what is conceptually equivalent, what can be obviously substituted and also what incorporates the essential idea of the embodiments.

What has been described above includes examples of one or more embodiments. It is, of course, not possible to describe every conceivable combination of components or methodologies for purposes of describing the aforementioned embodiments, but one of ordinary skill in the art may recognize that many further combinations and permutations of various embodiments are possible. Accordingly, the described embodiments are intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A method of tracking and analyzing behavioral data using a computing device, the method comprising:
receiving, at a processor associated with the computing device, a first input from a user to select a client profile;
receiving, at the processor associated with the computing device, a second input from the user to record event data associated with a behavioral event of a client;
recording, by the processor and to a memory associated with the computing device, the event data;
retrieving, by the processor and from the memory, historical behavioral data associated with the client profile;
compiling, by the processor based on the event data and the historical behavioral data, a summary of behavior associated with the client profile; and
displaying, on a display associated with the computing device, the summary of behavior associated with the client profile.

2. The method of claim 1, wherein receiving the first input and receiving the second input is performed by a processor associated with a wearable device.

3. The method of claim 1, wherein receiving the first input from the user comprises selecting the client profile from a user-configurable client list, wherein the user-configurable client list comprises a plurality of client profiles.

4. The method of claim 1, wherein the event data comprises time data indicative of a duration of the behavioral event.

5. The method of claim 1, wherein the event data comprises a description of the client's behavior during the behavioral event.

6. The method of claim 1, further comprising:
retrieving, by the processor and from the memory, behavioral plan data associated with one or more predetermined behavioral plans;
compiling, by the processor and based on the event data, the historical behavioral data, and the behavioral plan data, one or more client-specific data-based positive behavioral intervention plans; and
displaying, on the display, the one or more client-specific data-based positive behavioral intervention plans.

7. The method of claim 1, further comprising:
receiving, by the processor and from a positioning system associated with the computing device, location data associated with the behavioral event;
compiling, by the processor based on the event data and the location data, a summary of the behavioral event; and
displaying, on the display, the summary of the behavioral event.

8. The method of claim 1, further comprising:
receiving, at the processor, a third input from the user to record additional event data, the additional event data also being associated with the behavioral event of the client;
recording, in the memory, the additional event data; and
compiling, by the processor and based on the event data, the historical behavioral data, and the additional event data, the summary of behavior associated with the client profile.

9. The method of claim 8, wherein the third input from the user comprises selecting a severity level from a user-configurable list of severity levels descriptive of a severity of the behavioral event.

10. The method of claim 8, wherein the third input from the user comprises selecting an antecedent event from a user-configurable list of antecedent events descriptive of a cause of the behavioral event.

11. The method of claim 8, wherein the third input from the user comprises an event-specific note of the user.

12. The method of claim 8, wherein the third input from the user comprises selecting a consequence from a user-configurable list of consequences descriptive of a consequence of the behavioral event.

13. An apparatus, comprising:
a graphical user interface on a display, configured to present to a user, information of a summary of behavior associated with a client profile;
a processor in communication with the graphical user interface, configured to:
receive a first input from a user to select a client profile from a user-configurable client list;
receive a second input from the user to record a duration of a behavioral event of a client;
receive a third input from the user to select a behavioral description comprising a description of the client's behavior during the behavioral event;
receive a fourth input from the user to select an antecedent event from a user-configurable list of antecedent events descriptive of a cause of the behavioral event;
receive a fifth input from the user to select a consequence from a user-configurable list of consequences descriptive of a consequence of the behavioral event;
retrieve, from a memory in communication with the processor, historical behavioral data associated with the client profile;
compile, by the processor and based on the duration of the behavioral event, the behavioral description, the antecedent event, the consequence, and the historical behavioral data, a summary of behavior associated with the client profile; and
display, on the graphical user interface, the summary of behavior associated with the client profile.

14. The apparatus of claim 13, wherein the apparatus comprises a wearable device.

15. The apparatus of claim 13, wherein the apparatus further comprises a touch screen configured to receive a touch input from the user, and wherein at least one of the first input, the second input, the third input, the fourth input, and the fifth input comprises the touch input from the user.

16. The apparatus of claim 13, wherein the apparatus further comprises a sound recording device configured to receive a voice command from the user, wherein at least one of the first input, the second input, the third input, the fourth input, and the fifth input comprises the voice command from the user.

17. A system for tracking and analyzing behavioral data comprising:
a graphical user interface configured to receive an input from a user and display data associated with a client profile, wherein the graphical user interface comprises a wearable device;
a memory configured to store the data associated with a client profile;
a sound recording device configured to receive a voice command from a user;
a processor in communication with the graphical user interface, the memory, and the sound recording device, and configured to:
receive a first input from a user via the sound recording device to select a client profile from a user-configurable client list;
receive a second input from the user via the sound recording device to record a duration of a behavioral event of a client;
receive a third input from the user via the sound recording device to select a behavioral description comprising a description of the client's behavior during the behavioral event;
retrieve, from the memory, historical behavioral data associated with the client profile;
compile a summary of behavior associated with the client profile based on the duration, the behavioral description, and the historical behavioral data; and
display, on the graphical user interface, the summary of behavior associated with the client profile.

18. The system of claim 17, further comprising a connected computing device, wherein the processor is further configured to output the summary of behavior associated with the client profile to the connected computing device.

19. The system of claim 18, wherein the connected computing device comprises a remote server in communication with the processor.

20. The system of claim 17, further comprising a positioning system in communication with the processor, wherein the processor is further configured to:
receive, from the positioning system, location data associated with the behavioral event;
compile a summary of behavior associated with the client profile based on the duration, the behavioral description, the historical behavioral data, and the location data; and
display, on the graphical user interface, the summary of behavior associated with the client profile.
